(19) United States
(12) Patent Application Publication
(10) Pub. No.: US 2023/0008697 A1
(43) Pub. Date: Jan. 12, 2023

(54) MILLIMETER WAVE RADAR APPARATUS DETERMINING VITAL SIGN

(71) Applicant: WANSHIH ELECTRONIC CO., LTD., New Taipei City (TW)

(72) Inventors: Chun-Chi KO, New Taipei City (TW); Yi-Chen TSAI, New Taipei City (TW)

(21) Appl. No.: 17/372,509

(22) Filed: Jul. 11, 2021

(51) Int. Cl. A61B 5/0507; A61B 5/339; A61B 5/0245

(52) U.S. Cl. CPC A61B 5/0507; A61B 5/339; A61B 5/0245

(57) ABSTRACT

A millimeter wave radar apparatus determining a vital sign includes a microprocessor, a millimeter wave radar and an electrocardiogram machine. The millimeter wave radar is configured to detect a human body to obtain a plurality of wireless vital-sign signals. The microprocessor is configured to receive the wireless vital-sign signals. The electrocardiogram machine is configured to detect the human body to obtain a plurality of wired vital-sign signals. The microprocessor is configured to receive the wired vital-sign signals. After the microprocessor receives the wireless vital-sign signals and the wired vital-sign signals, the microprocessor is configured to output the wireless vital-sign signals and the wired vital-sign signals.

### BACKGROUND

### Technical Field

The present disclosure relates to a millimeter wave radar apparatus, and especially relates to a millimeter wave radar apparatus determining/detecting a vital sign.

### Description of Related Art

A related art electrocardiogram machine can determine/detect heartbeats of a human body, so as to determine/detect a basic health state of the human body. Therefore, the related art electrocardiogram machine is very important for human health. However, the current disadvantage of using the related art electrocardiogram machine to determine/detect the heartbeats of the human body is that there is no other apparatus to verify whether the heartbeats of the human body determined/detected by the related art electrocardiogram machine is accurate.

### SUMMARY OF THE DISCLOSURE

In order to solve the above-mentioned problems, an object of the present disclosure is to provide a millimeter wave radar apparatus determining/detecting a vital sign.

In order to achieve the object of the present disclosure mentioned above, the millimeter wave radar apparatus of the present disclosure is applied to a human body. The millimeter wave radar apparatus includes a microprocessor, a millimeter wave radar and an electrocardiogram machine. The millimeter wave radar is electrically connected to the microprocessor. The electrocardiogram machine is electrically connected to the microprocessor and the human body. Moreover, the millimeter wave radar is configured to determine/detect the human body to obtain a plurality of wireless vital-sign signals. After the millimeter wave radar obtains the wireless vital-sign signals, the microprocessor is configured to receive the wireless vital-sign signals. The electrocardiogram machine is configured to determine/detect the human body to obtain a plurality of wired vital-sign signals. After the electrocardiogram machine obtains the wired vital-sign signals, the microprocessor is configured to receive the wired vital-sign signals. After the microprocessor receives the wireless vital-sign signals and the wired vital-sign signals, the microprocessor is configured to output the wireless vital-sign signals and the wired vital-sign signals.

Moreover, in an embodiment of the millimeter wave radar apparatus of the present disclosure mentioned above, moreover the microprocessor is configured to synchronously output an n-th of the wireless vital-sign signals and the n-th of the wired vital-sign signals, wherein the n is an integer greater than zero.

Moreover, in an embodiment of the millimeter wave radar apparatus of the present disclosure mentioned above, moreover the microprocessor includes a data integration adjustment synchronization output unit electrically connected to the millimeter wave radar and the electrocardiogram machine, wherein the data integration adjustment synchronization output unit is configured to receive the wireless vital-sign signals and the wired vital-sign signals.

Moreover, in an embodiment of the millimeter wave radar apparatus of the present disclosure mentioned above, the millimeter wave radar apparatus further includes a display electrically connected to the data integration adjustment synchronization output unit, wherein after the data integration adjustment synchronization output unit receives the wireless vital-sign signals and the wired vital-sign signals, the data integration adjustment synchronization output unit is configured to synchronously output the n-th of the wireless vital-sign signals and the n-th of the wired vital-sign signals to the display, and the display is configured to synchronously receive the n-th of the wireless vital-sign signals and the n-th of the wired vital-sign signals, so that the display is configured to synchronously display the n-th of the wireless vital-sign signals and the n-th of the wired vital-sign signals.

Moreover, in an embodiment of the millimeter wave radar apparatus of the present disclosure mentioned above, the millimeter wave radar apparatus further includes a millimeter wave radar side communication interface and an electrocardiogram machine side communication interface. The millimeter wave radar side communication interface is electrically connected to the millimeter wave radar and the microprocessor. The electrocardiogram machine side communication interface is electrically connected to the electrocardiogram machine and the microprocessor. Moreover, the millimeter wave radar side communication interface is configured to receive the wireless vital-sign signals transmitted by the millimeter wave radar. The electrocardiogram machine side communication interface is configured to receive the wired vital-sign signals transmitted by the electrocardiogram machine.

Moreover, in an embodiment of the millimeter wave radar apparatus of the present disclosure mentioned above, moreover the microprocessor further includes a wireless vital-sign receiving unit electrically connected to the millimeter wave radar side communication interface, wherein after the millimeter wave radar side communication interface receives the wireless vital-sign signals, the millimeter wave radar side communication interface is configured to transmit the wireless vital-sign signals to the wireless vital-sign receiving unit. The wireless vital-sign receiving unit is configured to receive the wireless vital-sign signals transmitted by the millimeter wave radar side communication interface.

Moreover, in an embodiment of the millimeter wave radar apparatus of the present disclosure mentioned above, moreover the microprocessor further includes a wired vital-sign receiving unit electrically connected to the electrocardiogram machine side communication interface, wherein after the electrocardiogram machine side communication interface receives the wired vital-sign signals, the electrocardiogram machine side communication interface is configured to transmit the wired vital-sign signals to the wired vital-sign receiving unit. The wired vital-sign receiving unit is configured to receive the wired vital-sign signals transmitted by the electrocardiogram machine side communication interface.

Moreover, in an embodiment of the millimeter wave radar apparatus of the present disclosure mentioned above, moreover the microprocessor further includes a wireless vital-sign collecting unit electrically connected to the wireless vital-sign receiving unit and the data integration adjustment synchronization output unit, wherein after the wireless vital-sign receiving unit receives the wireless vital-sign signals, the wireless vital-sign receiving unit is configured to transmit the wireless vital-sign signals to the wireless vital-sign collecting unit. The wireless vital-sign collecting unit is configured to collect the wireless vital-sign signals transmitted by the wireless vital-sign receiving unit. After the wireless vital-sign collecting unit collects the wireless vital-sign signals, the wireless vital-sign collecting unit is configured to transmit the wireless vital-sign signals to the data integration adjustment synchronization output unit.

Moreover, in an embodiment of the millimeter wave radar apparatus of the present disclosure mentioned above, moreover the microprocessor further includes a wired vital-sign collecting unit electrically connected to the wired vital-sign receiving unit and the data integration adjustment synchronization output unit, wherein after the wired vital-sign receiving unit receives the wired vital-sign signals, the wired vital-sign receiving unit is configured to transmit the wired vital-sign signals to the wired vital-sign collecting unit. The wired vital-sign collecting unit is configured to collect the wired vital-sign signals transmitted by the wired vital-sign receiving unit. After the wired vital-sign collecting unit collects the wired vital-sign signals, the wired vital-sign collecting unit is configured to transmit the wired vital-sign signals to the data integration adjustment synchronization output unit.

Moreover, in an embodiment of the millimeter wave radar apparatus of the present disclosure mentioned above, moreover the millimeter wave radar side communication interface is a universal asynchronous receiver transmitter. The electrocardiogram machine side communication interface is a universal asynchronous receiver transmitter. The millimeter wave radar is configured to determine/detect a plurality of heartbeats of the human body to obtain the wireless vital-sign signals. The electrocardiogram machine is configured to determine/detect the heartbeats of the human body to obtain the wired vital-sign signals.

The advantage of the present disclosure is to utilize the millimeter wave radar to verify whether the heartbeats of the human body determined/detected by the electrocardiogram machine are accurate.

Please refer to the detailed descriptions and figures of the present disclosure mentioned below for further understanding the technology, method and effect of the present disclosure achieving the predetermined purposes. It believes that the purposes, characteristic and features of the present disclosure can be understood deeply and specifically. However, the figures are only for references and descriptions, but the present disclosure is not limited by the figures.

### BRIEF DESCRIPTION OF THE DRAWINGS

FIG. 1 shows a block diagram of an embodiment of the millimeter wave radar apparatus of the present disclosure.

FIG. 2 shows an application diagram of an embodiment of the millimeter wave radar apparatus of the present disclosure.

FIG. 3 shows a block diagram of an embodiment of the millimeter wave radar of the present disclosure.

FIG. 4 shows a block diagram of an embodiment of the analog-to-digital circuit of the present disclosure.

FIG. 5 shows a partial block diagram of an embodiment of the millimeter wave receiving circuit of the present disclosure.

FIG. 6 shows another partial block diagram of the embodiment of the millimeter wave receiving circuit of the present disclosure.

FIG. 7 shows a block diagram of an embodiment of the millimeter wave transmitting circuit of the present disclosure.

### DETAILED DESCRIPTION

In the present disclosure, numerous specific details are provided, to provide a thorough understanding of embodiments of the disclosure. Persons of ordinary skill in the art will recognize, however, that the present disclosure can be practiced without one or more of the specific details. In other instances, well-known details are not shown or described to avoid obscuring aspects of the present disclosure. Now please refer to the figures for the explanation of the technical content and the detailed description of the present disclosure:

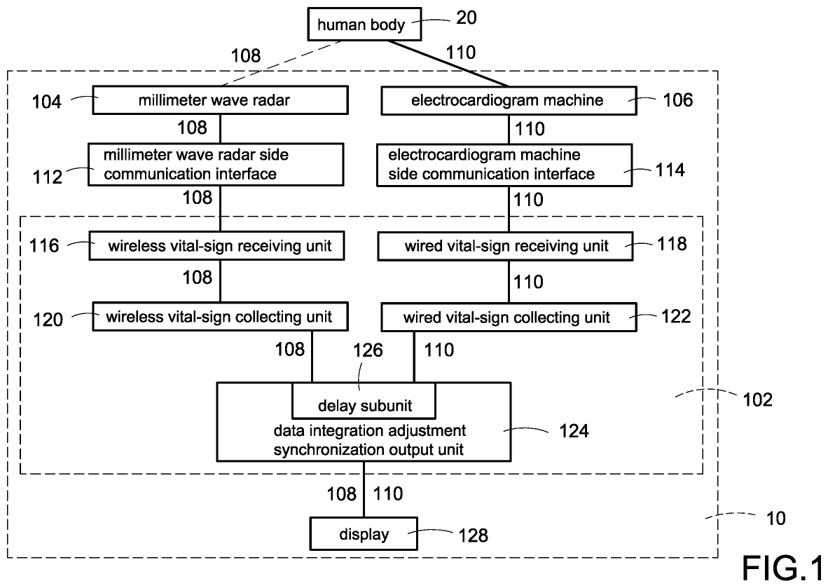

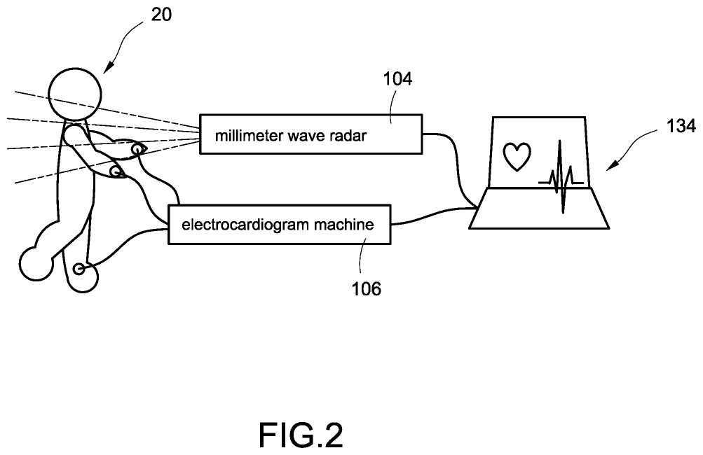

FIG. 1 shows a block diagram of an embodiment of the millimeter wave radar apparatus of the present disclosure. FIG. 2 shows an application diagram of an embodiment of the millimeter wave radar apparatus of the present disclosure. A millimeter wave radar apparatus 10 determining/detecting a vital sign of the present disclosure includes a microprocessor 102, a millimeter wave radar 104, an electrocardiogram machine 106, a millimeter wave radar side communication interface 112, an electrocardiogram machine side communication interface 114 and a display 128. The microprocessor 102 includes a wireless vital-sign receiving unit 116, a wired vital-sign receiving unit 118, a wireless vital-sign collecting unit 120, a wired vital-sign collecting unit 122 and a data integration adjustment synchronization output unit 124. The components mentioned above are electrically connected to each other.

It should be noted that the present disclosure only needs the microprocessor 102, the millimeter wave radar 104 and the electrocardiogram machine 106 to achieve the effect and the purpose of the present disclosure. Except the millimeter wave radar 104 and the electrocardiogram machine 106, the other components of the millimeter wave radar apparatus 10 of the present disclosure mentioned above can be integrated into a computer 134. The millimeter wave radar apparatus 10 of the present disclosure is applied to a human body 20.

The millimeter wave radar 104 is configured to determine/detect the human body 20 to obtain a plurality of wireless vital-sign signals 108. After the millimeter wave radar 104 obtains the wireless vital-sign signals 108, the microprocessor 102 is configured to receive the wireless vital-sign signals 108. The electrocardiogram machine 106 is configured to determine/detect the human body 20 to obtain a plurality of wired vital-sign signals 110. After the electrocardiogram machine 106 obtains the wired vital-sign signals 110, the microprocessor 102 is configured to receive the wired vital-sign signals 110. After the microprocessor 102 receives the wireless vital-sign signals 108 and the wired vital-sign signals 110, the microprocessor 102 is configured to synchronously output an n-th of the wireless vital-sign signals 108 and the n-th of the wired vital-sign signals 110, wherein the n is an integer greater than zero, namely, 1, 2, 3 and so on.

This is because the vital-sign signals determined/detected and generated by different apparatuses may not be synchronized (or may be synchronized). For example, due to the difference in signal processing time, the vital-sign signals determined/detected and generated by a certain apparatus within a fixed time is less than the vital-sign signals determined/detected and generated by another apparatus, which is called out of synchronization. For example, a certain apparatus determines/detects and generates 10 vital-sign signals within the fixed time, but another apparatus determines/detects and generates 20 vital-sign signals within the fixed time. If all the vital-sign signals are outputted in the fixed time, a certain apparatus will only output and display 10 vital-sign signals, but another apparatus will output and display 20 vital-sign signals; this will cause out-of-sync chaos.

For the present disclosure, the wireless vital-sign signals 108 determined/detected by the millimeter wave radar 104 and the wired vital-sign signals 110 determined/detected by the electrocardiogram machine 106 may not be synchronized (or may be synchronized). If the wireless vital-sign signals 108 determined/detected by the millimeter wave radar 104 and the wired vital-sign signals 110 determined/detected by the electrocardiogram machine 106 are not synchronized, the microprocessor 102 provided by the present disclosure is configured to be able to wait and synchronously (simultaneously) output the first of the wireless vital-sign signals 108 and the first of the wired vital-sign signals 110, and then wait and synchronously (simultaneously) output the second of the wireless vital-sign signals 108 and the second of the wired vital-sign signals 110, and then wait and synchronously (simultaneously) output the third of the wireless vital-sign signals 108 and the third of the wired vital-sign signals 110, and so on. Therefore, the present disclosure will not cause out-of-sync chaos.

The millimeter wave radar side communication interface 112 is configured to receive the wireless vital-sign signals 108 transmitted by the millimeter wave radar 104. The electrocardiogram machine side communication interface 114 is configured to receive the wired vital-sign signals 110 transmitted by the electrocardiogram machine 106.

After the millimeter wave radar side communication interface 112 receives the wireless vital-sign signals 108, the millimeter wave radar side communication interface 112 is configured to transmit the wireless vital-sign signals 108 to the wireless vital-sign receiving unit 116. The wireless vital-sign receiving unit 116 is configured to receive the wireless vital-sign signals 108 transmitted by the millimeter wave radar side communication interface 112.

After the electrocardiogram machine side communication interface 114 receives the wired vital-sign signals 110, the electrocardiogram machine side communication interface 114 is configured to transmit the wired vital-sign signals 110 to the wired vital-sign receiving unit 118. The wired vital-sign receiving unit 118 is configured to receive the wired vital-sign signals 110 transmitted by the electrocardiogram machine side communication interface 114.

After the wireless vital-sign receiving unit 116 receives the wireless vital-sign signals 108, the wireless vital-sign receiving unit 116 is configured to transmit the wireless vital-sign signals 108 to the wireless vital-sign collecting unit 120. The wireless vital-sign collecting unit 120 is configured to collect the wireless vital-sign signals 108 transmitted by the wireless vital-sign receiving unit 116. After the wireless vital-sign collecting unit 120 collects the wireless vital-sign signals 108, the wireless vital-sign collecting unit 120 is configured to transmit the wireless vital-sign signals 108 to the data integration adjustment synchronization output unit 124.

After the wired vital-sign receiving unit 118 receives the wired vital-sign signals 110, the wired vital-sign receiving unit 118 is configured to transmit the wired vital-sign signals 110 to the wired vital-sign collecting unit 122. The wired vital-sign collecting unit 122 is configured to collect the wired vital-sign signals 110 transmitted by the wired vital-sign receiving unit 118. After the wired vital-sign collecting unit 122 collects the wired vital-sign signals 110, the wired vital-sign collecting unit 122 is configured to transmit the wired vital-sign signals 110 to the data integration adjustment synchronization output unit 124.

The data integration adjustment synchronization output unit 124 is configured to receive the wireless vital-sign signals 108 and the wired vital-sign signals 110. After the data integration adjustment synchronization output unit 124 receives the wireless vital-sign signals 108 and the wired vital-sign signals 110, the data integration adjustment synchronization output unit 124 is configured to synchronously (simultaneously) output the n-th of the wireless vital-sign signals 108 and the n-th of the wired vital-sign signals 110 to the display 128, and the display 128 is configured to synchronously receive the n-th of the wireless vital-sign signals 108 and the n-th of the wired vital-sign signals 110, so that the display 128 is configured to synchronously (simultaneously) display the n-th of the wireless vital-sign signals 108 and the n-th of the wired vital-sign signals 110.

The millimeter wave radar side communication interface 112 is, for example but not limited to, a universal asynchronous receiver transmitter. The electrocardiogram machine side communication interface 114 is, for example but not limited to, a universal asynchronous receiver transmitter. The millimeter wave radar 104 is configured to determine/detect a plurality of heartbeats of the human body 20 to obtain the wireless vital-sign signals 108. The electrocardiogram machine 106 is configured to determine/detect the heartbeats of the human body 20 to obtain the wired vital-sign signals 110.

Moreover, the data integration adjustment synchronization output unit 124 includes a delay subunit 126 electrically connected to the millimeter wave radar 104 and the electrocardiogram machine 106. After the data integration adjustment synchronization output unit 124 receives the wireless vital-sign signals 108 and the wired vital-sign signals 110, the data integration adjustment synchronization output unit 124 is configured to utilize the delay subunit 126 to delay the wireless vital-sign signals 108 or the wired vital-sign signals 110 (namely, to delay the faster signals), so as to synchronously (simultaneously) output the n-th of the wireless vital-sign signals 108 and the n-th of the wired vital-sign signals 110.

The wireless vital-sign receiving unit 116, the wired vital-sign receiving unit 118, the wireless vital-sign collecting unit 120, the wired vital-sign collecting unit 122, the data integration adjustment synchronization output unit 124 and the delay subunit 126 can be integrated into the microprocessor 102. Namely, the respective works of the above-mentioned units/subunits are all performed by the microprocessor 102. Or, the above-mentioned units/subunits are respective microprocessors or signal processors or electronic components, so as to perform the respective works of the above-mentioned units/subunits.

For example, the wireless vital-sign receiving unit 116 is a first microprocessor, a first signal processor or a first signal transceiver; the wired vital-sign receiving unit 118 is a second microprocessor, a second signal processor or a second signal transceiver; the wireless vital-sign collecting unit 120 is a third microprocessor or a third signal processor; the wired vital-sign collecting unit 122 is a fourth microprocessor or a fourth signal processor; the data integration adjustment synchronization output unit 124 is a fifth microprocessor or a fifth signal processor; the delay subunit 126 is a sixth microprocessor, a sixth signal processor or a delayer.

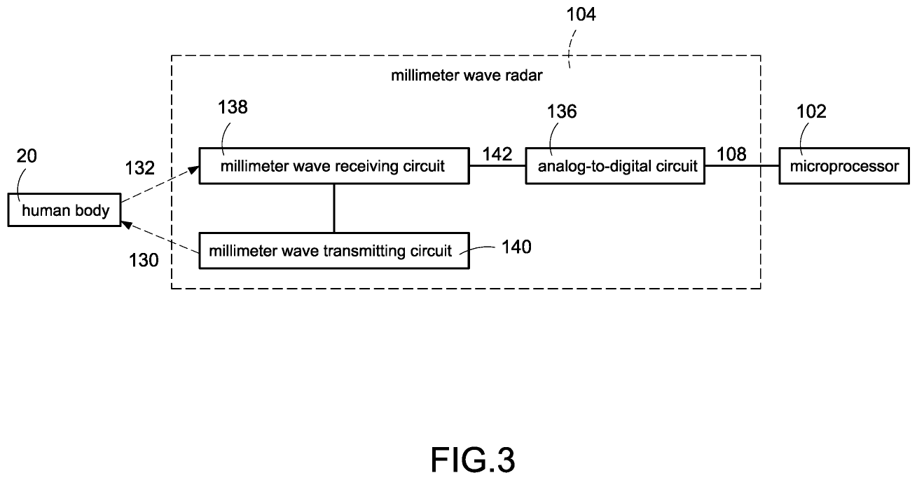

Moreover, FIG. 3 shows a block diagram of an embodiment of the millimeter wave radar of the present disclosure. Please refer to FIG. 1 to FIG. 2 together. The millimeter wave radar 104 includes an analog-to-digital circuit 136, a millimeter wave receiving circuit 138 and a millimeter wave transmitting circuit 140. The analog-to-digital circuit 136 is electrically connected to the microprocessor 102. The millimeter wave receiving circuit 138 is electrically connected to the analog-to-digital circuit 136. The millimeter wave transmitting circuit 140 is electrically connected to the millimeter wave receiving circuit 138. The millimeter wave transmitting circuit 140 is configured to transmit a radar wave 130 to the human body 20. The millimeter wave receiving circuit 138 is configured to receive a reflected radar wave 132 reflected from the human body 20 based on the radar wave 130. The millimeter wave receiving circuit 138 is configured to process the reflected radar wave 132 to obtain an analog signal 142. The millimeter wave receiving circuit 138 is configured to transmit the analog signal 142 to the analog-to-digital circuit 136. The analog-to-digital circuit 136 is configured to process the analog signal 142 to obtain the wireless vital-sign signals 108. The analog-to-digital circuit 136 is configured to transmit the wireless vital-sign signals 108 to the microprocessor 102.

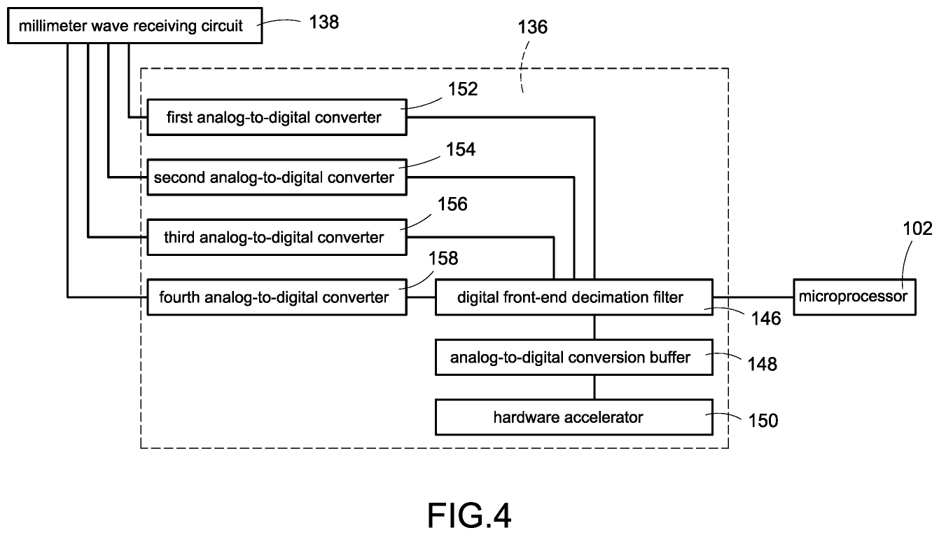

Moreover, FIG. 4 shows a block diagram of an embodiment of the analog-to-digital circuit of the present disclosure. Please refer to FIG. 1 to FIG. 3 together. The analog-to-digital circuit 136 includes a digital front-end decimation filter 146, an analog-to-digital conversion buffer 148, a hardware accelerator 150, a first analog-to-digital converter 152, a second analog-to-digital converter 154, a third analog-to-digital converter 156 and a fourth analog-to-digital converter 158. The digital front-end decimation filter 146 is electrically connected to the microprocessor 102. The analog-to-digital conversion buffer 148 is electrically connected to the digital front-end decimation filter 146. The hardware accelerator 150 is electrically connected to the analog-to-digital conversion buffer 148. The first analog-to-digital converter 152 is electrically connected to the digital front-end decimation filter 146 and the millimeter wave receiving circuit 138. The second analog-to-digital converter 154 is electrically connected to the digital front-end decimation filter 146 and the millimeter wave receiving circuit 138. The third analog-to-digital converter 156 is electrically connected to the digital front-end decimation filter 146 and the millimeter wave receiving circuit 138. The fourth analog-to-digital converter 158 is electrically connected to the digital front-end decimation filter 146 and the millimeter wave receiving circuit 138.

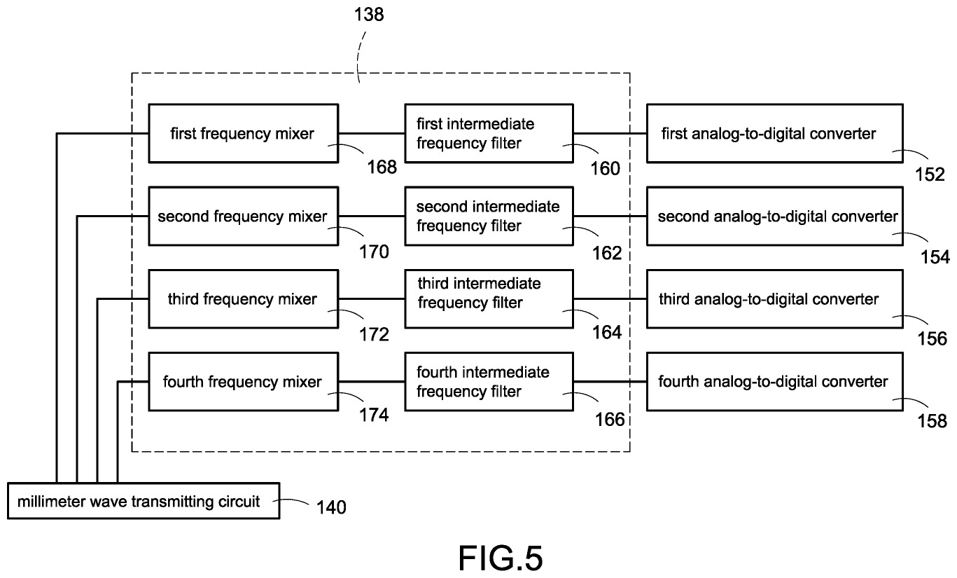

Moreover, FIG. 5 shows a partial block diagram of an embodiment of the millimeter wave receiving circuit of the present disclosure. Please refer to FIG. 1 to FIG. 4 together. The millimeter wave receiving circuit 138 includes a first intermediate frequency filter 160, a second intermediate frequency filter 162, a third intermediate frequency filter 164, a fourth intermediate frequency filter 166, a first frequency mixer 168, a second frequency mixer 170, a third frequency mixer 172 and a fourth frequency mixer 174. The first intermediate frequency filter 160 is electrically connected to the first analog-to-digital converter 152. The second intermediate frequency filter 162 is electrically connected to the second analog-to-digital converter 154. The third intermediate frequency filter 164 is electrically connected to the third analog-to-digital converter 156. The fourth intermediate frequency filter 166 is electrically connected to the fourth analog-to-digital converter 158. The first frequency mixer 168 is electrically connected to the first intermediate frequency filter 160 and the millimeter wave transmitting circuit 140. The second frequency mixer 170 is electrically connected to the second intermediate frequency filter 162 and the millimeter wave transmitting circuit 140. The third frequency mixer 172 is electrically connected to the third intermediate frequency filter 164 and the millimeter wave transmitting circuit 140. The fourth frequency mixer 174 is electrically connected to the fourth intermediate frequency filter 166 and the millimeter wave transmitting circuit 140.

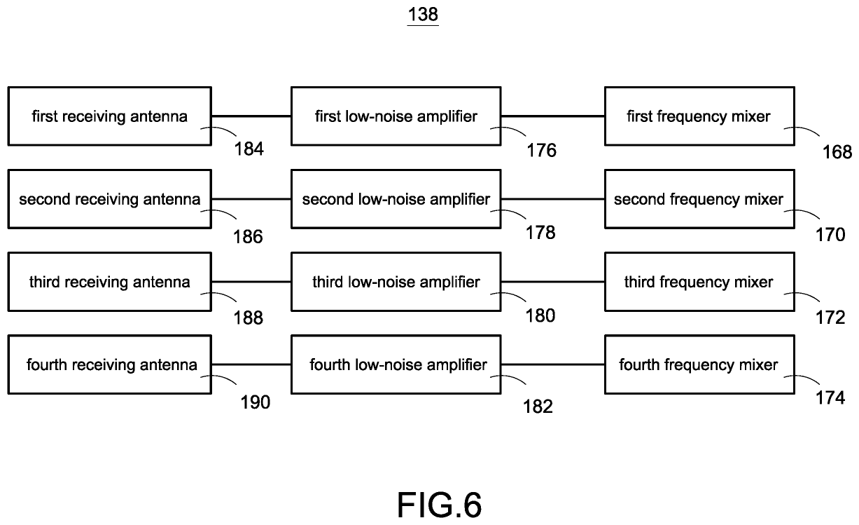

Moreover, FIG. 6 shows another partial block diagram of the embodiment of the millimeter wave receiving circuit of the present disclosure. Please refer to FIG. 1 to FIG. 5 together. The millimeter wave receiving circuit 138 further includes a first low-noise amplifier 176, a second low-noise amplifier 178, a third low-noise amplifier 180, a fourth low-noise amplifier 182, a first receiving antenna 184, a second receiving antenna 186, a third receiving antenna 188 and a fourth receiving antenna 190. The first low-noise amplifier 176 is electrically connected to the first frequency mixer 168. The second low-noise amplifier 178 is electrically connected to the second frequency mixer 170. The third low-noise amplifier 180 is electrically connected to the third frequency mixer 172. The fourth low-noise amplifier 182 is electrically connected to the fourth frequency mixer 174. The first receiving antenna 184 is electrically connected to the first low-noise amplifier 176. The second receiving antenna 186 is electrically connected to the second low-noise amplifier 178. The third receiving antenna 188 is electrically connected to the third low-noise amplifier 180. The fourth receiving antenna 190 is electrically connected to the fourth low-noise amplifier 182.

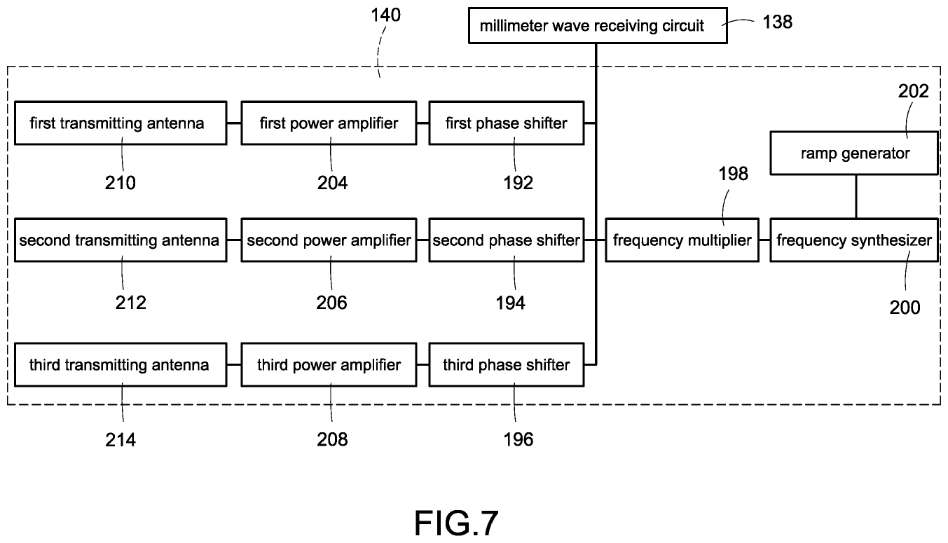

Moreover, FIG. 7 shows a block diagram of an embodiment of the millimeter wave transmitting circuit of the present disclosure. Please refer to FIG. 1 to FIG. 6 together. The millimeter wave transmitting circuit 140 includes a first phase shifter 192, a second phase shifter 194, a third phase shifter 196, a frequency multiplier 198, a frequency synthesizer 200 and a ramp generator 202. The first phase shifter 192 is electrically connected to the millimeter wave receiving circuit 138. The second phase shifter 194 is electrically connected to the millimeter wave receiving circuit 138. The third phase shifter 196 is electrically connected to the millimeter wave receiving circuit 138. The frequency multiplier 198 is electrically connected to the millimeter wave receiving circuit 138, the first phase shifter 192, the second phase shifter 194 and the third phase shifter 196. The frequency synthesizer 200 is electrically connected to the frequency multiplier 198. The ramp generator 202 is electrically connected to the frequency synthesizer 200.

Moreover, according to FIG. 7, the millimeter wave transmitting circuit 140 further includes a first power amplifier 204, a second power amplifier 206, a third power amplifier 208, a first transmitting antenna 210, a second transmitting antenna 212 and a third transmitting antenna 214. The first power amplifier 204 is electrically connected to the first phase shifter 192. The second power amplifier 206 is electrically connected to the second phase shifter 194. The third power amplifier 208 is electrically connected to the third phase shifter 196. The first transmitting antenna 210 is electrically connected to the first power amplifier 204. The second transmitting antenna 212 is electrically connected to the second power amplifier 206. The third transmitting antenna 214 is electrically connected to the third power amplifier 208.

The advantage of the present disclosure is to utilize the millimeter wave radar to verify whether the heartbeats of the human body determined/detected by the electrocardiogram machine are accurate. Moreover, a number of the heartbeats of a person in one minute is a fixed fact, so different apparatuses determining/detecting it shall obtain the similar data, but because the signal processing time of each apparatus is different, some data is generated fast but some data is generated slow. The present disclosure can utilize the delay mechanism/technique to make the faster-generated data wait for the slower-generated data, so as to simultaneously display the first data of different apparatuses, and then simultaneously display the second data of different apparatuses, and then simultaneously display the third data of different apparatuses, and so on. Therefore, the present disclosure will not cause out-of-sync chaos.

Moreover, the millimeter wave radar apparatus 10 further includes a camera (not shown in these figures). The camera is electrically connected to the data integration adjustment synchronization output unit 124. If the millimeter wave radar 104 or the electrocardiogram machine 106 detects that the wireless vital-sign signals 108 or the wired vital-sign signals 110 of the human body 20 are abnormal, the camera is configured to be turned on to photograph the human body 20 to display on a camera display screen (not shown in these figures) of the display 128.

Although the present disclosure has been described with reference to the preferred embodiment thereof, it will be understood that the disclosure is not limited to the details thereof. Various substitutions and modifications have been suggested in the foregoing description, and others will occur to those of ordinary skill in the art. Therefore, all such substitutions and modifications are intended to be embraced within the scope of the disclosure as defined in the appended claims.

What is claimed is:

1. A millimeter wave radar apparatus determining a vital sign applied to a human body, the millimeter wave radar apparatus comprising:
a microprocessor;
a millimeter wave radar electrically connected to the microprocessor; and
an electrocardiogram machine electrically connected to the microprocessor and the human body,
wherein the millimeter wave radar is configured to detect the human body to obtain a plurality of wireless vital-sign signals; after the millimeter wave radar obtains the wireless vital-sign signals, the microprocessor is configured to receive the wireless vital-sign signals; the electrocardiogram machine is configured to detect the human body to obtain a plurality of wired vital-sign signals; after the electrocardiogram machine obtains the wired vital-sign signals, the microprocessor is configured to receive the wired vital-sign signals; after the microprocessor receives the wireless vital-sign signals and the wired vital-sign signals, the microprocessor is configured to output the wireless vital-sign signals and the wired vital-sign signals.

2. The millimeter wave radar apparatus of claim 1, wherein the microprocessor is configured to synchronously output an n-th of the wireless vital-sign signals and the n-th of the wired vital-sign signals, wherein the n is an integer greater than zero.

3. The millimeter wave radar apparatus of claim 2, wherein the microprocessor comprises:
a data integration adjustment synchronization output unit electrically connected to the millimeter wave radar and the electrocardiogram machine,
wherein the data integration adjustment synchronization output unit is configured to receive the wireless vital-sign signals and the wired vital-sign signals.

4. The millimeter wave radar apparatus of claim 3, further comprising:
a display electrically connected to the data integration adjustment synchronization output unit,
wherein after the data integration adjustment synchronization output unit receives the wireless vital-sign signals and the wired vital-sign signals, the data integration adjustment synchronization output unit is configured to synchronously output the n-th of the wireless vital-sign signals and the n-th of the wired vital-sign signals to the display, and the display is configured to synchronously receive the n-th of the wireless vital-sign signals and the n-th of the wired vital-sign signals, so that the display is configured to synchronously display the n-th of the wireless vital-sign signals and the n-th of the wired vital-sign signals.

5. The millimeter wave radar apparatus of claim 4, further comprising:
a millimeter wave radar side communication interface electrically connected to the millimeter wave radar and the microprocessor; and
an electrocardiogram machine side communication interface electrically connected to the electrocardiogram machine and the microprocessor,
wherein the millimeter wave radar side communication interface is configured to receive the wireless vital-sign signals transmitted by the millimeter wave radar; the electrocardiogram machine side communication interface is configured to receive the wired vital-sign signals transmitted by the electrocardiogram machine.

6. The millimeter wave radar apparatus of claim 5, wherein the microprocessor further comprises:
a wireless vital-sign receiving unit electrically connected to the millimeter wave radar side communication interface,
wherein after the millimeter wave radar side communication interface receives the wireless vital-sign signals, the millimeter wave radar side communication interface is configured to transmit the wireless vital-sign signals to the wireless vital-sign receiving unit; the wireless vital-sign receiving unit is configured to receive the wireless vital-sign signals transmitted by the millimeter wave radar side communication interface.

7. The millimeter wave radar apparatus of claim 6, wherein the microprocessor further comprises:
a wired vital-sign receiving unit electrically connected to the electrocardiogram machine side communication interface,
wherein after the electrocardiogram machine side communication interface receives the wired vital-sign signals, the electrocardiogram machine side communication interface is configured to transmit the wired vital-sign signals to the wired vital-sign receiving unit; the wired vital-sign receiving unit is configured to receive the wired vital-sign signals transmitted by the electrocardiogram machine side communication interface.

8. The millimeter wave radar apparatus of claim 7, wherein the microprocessor further comprises:
a wireless vital-sign collecting unit electrically connected to the wireless vital-sign receiving unit and the data integration adjustment synchronization output unit,
wherein after the wireless vital-sign receiving unit receives the wireless vital-sign signals, the wireless vital-sign receiving unit is configured to transmit the wireless vital-sign signals to the wireless vital-sign collecting unit; the wireless vital-sign collecting unit is configured to collect the wireless vital-sign signals transmitted by the wireless vital-sign receiving unit; after the wireless vital-sign collecting unit collects the wireless vital-sign signals, the wireless vital-sign collecting unit is configured to transmit the wireless vital-sign signals to the data integration adjustment synchronization output unit.

9. The millimeter wave radar apparatus of claim 8, wherein the microprocessor further comprises:
a wired vital-sign collecting unit electrically connected to the wired vital-sign receiving unit and the data integration adjustment synchronization output unit,
wherein after the wired vital-sign receiving unit receives the wired vital-sign signals, the wired vital-sign receiving unit is configured to transmit the wired vital-sign signals to the wired vital-sign collecting unit; the wired vital-sign collecting unit is configured to collect the wired vital-sign signals transmitted by the wired vital-sign receiving unit; after the wired vital-sign collecting unit collects the wired vital-sign signals, the wired vital-sign collecting unit is configured to transmit the wired vital-sign signals to the data integration adjustment synchronization output unit.

10. The millimeter wave radar apparatus of claim 9, wherein the millimeter wave radar side communication interface is a universal asynchronous receiver transmitter; the electrocardiogram machine side communication interface is a universal asynchronous receiver transmitter; the millimeter wave radar is configured to detect a plurality of heartbeats of the human body to obtain the wireless vital-sign signals; the electrocardiogram machine is configured to detect the heartbeats of the human body to obtain the wired vital-sign signals.
